(19) United States
(12) Patent Application Publication
(10) Pub. No.: US 2008/0076102 A1
(43) Pub. Date: Mar. 27, 2008

(54) Method for Providing an InVivo Model of Disc Degeneration

(75) Inventor: Miguelangelo J. Perez-Cruet, Bloomfield, MI (US)

Correspondence Address: MILLER IP GROUP, PLC;MI4 SPINE, LLC, 42690 WOODWARD AVE., SUITE 200, BLOOMFIELD HILLS, MI, 48304, US

(73) Assignee: MI4SPINE, LLC, Bloomfield Village, MI (US)

(21) Appl. No.: 11/756,274

(22) Filed: May 31, 2007

Related U.S. Application Data
(60) Provisional application No. 60/846,437, filed on Sep. 22, 2006.

(51) Int. Cl. G09B 23/28; A61B 5/05

(52) U.S. Cl. 434/274; 600/407

(57) ABSTRACT

A method for providing an InVivo model of disc degeneration. The method includes percutaneously inserting a needle into a non-human surrogate disc, generally the disc of a rabbit, so that the needle ruptures the annulus of the disc. Fluoroscopy X-ray images can be used to visualize the placement of the needle to the proper location. By using the proper surrogate, rupture of the annulus of the disc will have a quick degenerative effect on the disc, which can be visualized using MRI or other suitable imaging devices. By studying the etiologies associated with disc degeneration of the surrogate, treatments can then be devised to attempt to reduce the rate of degeneration, reverse degeneration, or other disc treatment procedures.

### CROSS-REFERENCE TO RELATED APPLICATIONS

This application claims priority to U.S. Provisional Patent Application Ser. No. 60/846,437, filed Sep. 22, 2006 and titled “Method for Providing an InVivo Model of Disc Degeneration.”

### BACKGROUND OF THE INVENTION

1. Field of the Invention

This invention relates generally to a method for providing an InVivo model of disc degeneration and, more particularly, to a method for percutaneously rupturing an intervertebral disc of an animal using a needle so that degeneration of the disc can be studied.

2. Discussion of the Related Art

The human spine includes a series of vertebrae interconnected by connective tissue referred to as discs that act as a cushion between the vertebrae. The discs allow for movement of the vertebrae so that the back can bend and rotate.

The intervertebral disc is an active organ in which the normal and pathologic anatomies are well known, but the normal and pathologic physiologies have not been greatly understood. The intervertebral disc permits rhythmic motions required of all vertebrate animals in their various forms of locomotion. The disc is a high-pressure system composed primarily of absorbed water, an outer multilayered circumferential annulus of strong, flexible, but essentially inelastic collagen fibers, and an inner core of a hydrogel. The swelling of the contained hydrogel creates the high pressure that tightens the annular fibers and its laminations. Degeneration of discs in humans is typically a slow, complex process involving essentially all of the mechanical and physiologic components. Discogenic pain arises from either component, but is primarily due to altered chemistry. When this pain is severely disabling and unyielding, the preferred contemporary treatments are primarily surgical, particularly fusion and/or disc replacement.

Annular collagen fibers are arranged in circumferential belts or laminations inserting strongly and tangentially in right- and left-handed angulated patches into each adjacent vertebral body. Inside the annular ring is contained an aggrecan, glycosaminoglycan, a protein-sugar complex gel having great hygroscopic ability, i.e., water holding capacity. The swelling pressure of this gel of the nucleus maintains the pressure within the annulus, forcing the vertebrae apart and tightening the annular fibers. This tightening provides the primary mechanical stability and flexibility of each disc of the spinal column. Further, the angulated arrangement of the fibers also controls the segmental stability and flexibility of the motion segment. Therefore, the motion of each segment relates directly to the swelling capacity of the gel and secondarily to the tightness of intact annulus fibers. The same gel is also found in thin layers separating the annular laminar construction, providing some apparent elasticity and separating the laminations, reducing interlaminar torsional abrasion. With aging or degeneration, nucleus gel declines, while collagen content, including fibrosis, relatively increases.

Disc degeneration, which involves matrix, collagen and aggrecan, usually begins with annular tears or alterations in the endplate nutritional pathways by mechanical or pathophysiologic means. However, the disc ultimately fails for cellular reasons. It is believed that at an early age the central core of the disc, the nucleus pulposus, is made up of notochordal cells. These cells lead to the formation of the spinal column and the intervertebral disc. The notochordal cells help to create a proteoglycan matrix that holds water and supports the weight of the vertebral column. As one ages, typically after about 10 years in humans, there is a loss of the notochordal cells within the disc. As these cells are lost, they are replaced by chondrocytes that make up the mature nucleus pulposus.

There is also a relative decline in the proteoglycan matrix that holds water. Therefore, the disc begins to dry out or desiccate. As this process progresses, the disc loses its height and water holding capacity, and the disc degeneration process begins. The outer fibers of the disc starts to get annular tears, leading to further disc degeneration and desiccation. As the disc collapses, the nerves can get progressively compressed or pinched as they leave the spine, resulting in back pain conditions. Additionally, the back pain can result from disc degeneration itself without nerve compression. This condition is not entirely understood and results in tremendous health dollar expenditures and loss of worker productivity. Currently, there are no treatment options available to slow down, impede or stop disc degeneration, and it remains a part of the aging process of the intervertebral disc.

Progressive injury and aging of the disc occurs normally in later life and abnormally after trauma or metabolic changes. In addition to the chemical effects on the free nerve endings as a source of discogenic pain, other factors may lead to chronic back pain disorders. Free nerve endings in the annular fibers may be stimulated by stretching as the disc degenerates, bulges, and circumferential delamination of annular fibers occurs.

A model for studying the process of disc degeneration is critical in understanding the process and developing treatment options. It would be desirable to provide a non-human model through which intervertebral disc degeneration can be studied so that various treatments can be developed that help treat, rejuvenate, slow down, etc. natural or non-natural human disc degeneration.

### SUMMARY OF THE INVENTION

In accordance with the teachings of the present invention, a method for providing an InVivo model of disc degeneration is disclosed. The method includes percutaneously inserting a needle into a non-human surrogate disc, generally the disc of a rabbit, so that the needle ruptures the annulus of the disc. Fluoroscopy X-ray images can be used to visualize the placement of the needle to the proper location within the center of the disc. By using the proper surrogate, rupture of the annulus of the disc will have a quick degenerative effect on the disc, which can be visualized using magnetic resonance imaging (MRI) or other suitable imaging devices. By studying the etiologies associated with disc degeneration of the surrogate, treatments can then be devised to attempt to reduce the rate of degeneration, reverse degeneration, or other disc treatment procedures.

Additional features of the present invention will become apparent from the following description and appended claims, taken in conjunction with the accompanying drawings.

### BRIEF DESCRIPTION OF THE DRAWINGS

FIG. 1 is a perspective view of a needle being percutaneously inserted into a disc of an anesthetized rabbit;

FIG. 2 is a close-up view of the needle inserted into the disc of the rabbit;

FIG. 3 is a fluoroscopic image of a needle being positioned into the disc of a rabbit;

FIG. 4 is a plan view of the rabbit being imaged by an MRI device;

FIG. 5 is an MRI image of a rabbit showing InVivo created disc degeneration in a rabbit spine model and normal appearing rabbit intervertebral discs; and

FIG. 6 is an MRI of a degenerated disc at L₅S₁ in a human spine.

### DETAILED DESCRIPTION OF THE EMBODIMENTS

The following discussion of the embodiments of the invention directed to a method for providing an InVivo model of disc degeneration is merely exemplary in nature, and is in no way intended to limit the invention or its applications or uses.

The present invention proposes a process for providing an InVivo model of disc degeneration, where the disc degeneration occurs relatively rapidly in the model, and can be easily studied, to learn the processes of human disc degeneration, which typically occurs slowly.

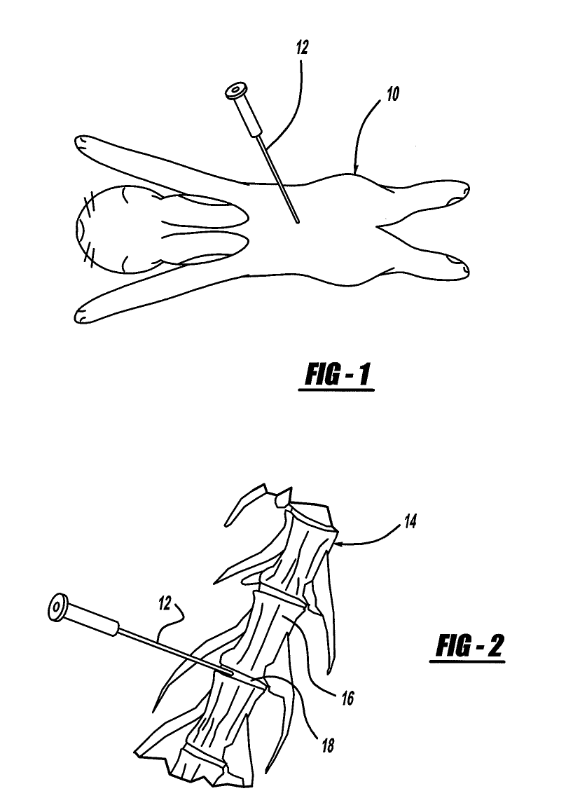

FIG. 1 is a perspective view of a rabbit 10 that has been anesthetized, where one or more of the intervertebral discs in the rabbit 10 will be the InVivo model. A technician will percutaneously insert, i.e., through the skin, a needle 12 into the rabbit 10 towards an intervertebral disc of the rabbit 10. FIG. 2 is a perspective view of a spine 14 of the rabbit 10 including vertebrae 16 and discs 18 therebetween. The needle 12 is shown inserted into one of the discs 18 of the rabbit's vertebrae 16, where the needle 12 ruptures the annulus of the disc 18. By rupturing the disc in this fashion, the inner gelatinous portion of the disc begins to dry out and desiccate causing the disc to degenerate. In this non-limiting embodiment, the needle 12 is a 16-gage needle because it is a proper size for the height of the disc 18. However, in other embodiments, other needle sizes may be more applicable.

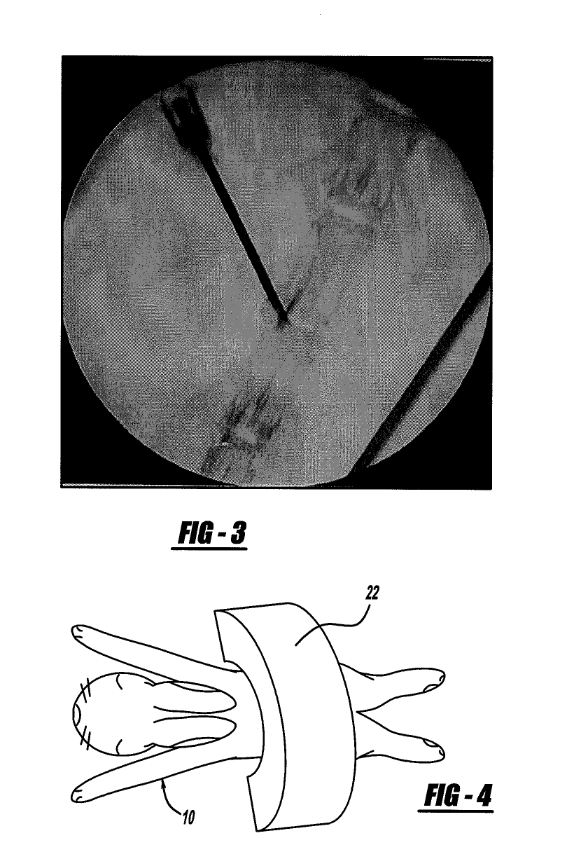

The technician can use fluoroscopic X-ray images to allow the technician to visualize the location of the needle 12 in the rabbit 10 so that the technician is able to properly position the needle 12, as shown. FIG. 3 is a fluoroscopic X-ray image showing a needle positioned within the disc of a rabbit for the purposes described herein. Other methods for guiding the needle 12 can also be employed, such as x-rays, computer tomography, tomograms, MRIs, etc.

By puncturing the annulus of the disc in an animal model, disc degeneration will begin to occur. This procedure may also be used to induce disc degeneration in other animal models, including primates. For humans, disc degeneration from old age or disc damage occurs relatively slowly over several years. However, with a suitable animal model, disc degeneration occurs much more rapidly, typically on the order of a few weeks. Although other lab animals can be used as the model, such as rats, mice, sheep, primates, etc., rabbits are used as the more preferable lab specimen because they are relatively easy to work with and are of a large enough size. Further, because a rabbit has a relatively upright posture when sitting, it mimics the loading of the lower spine intervertebral disc that is similar to a human.

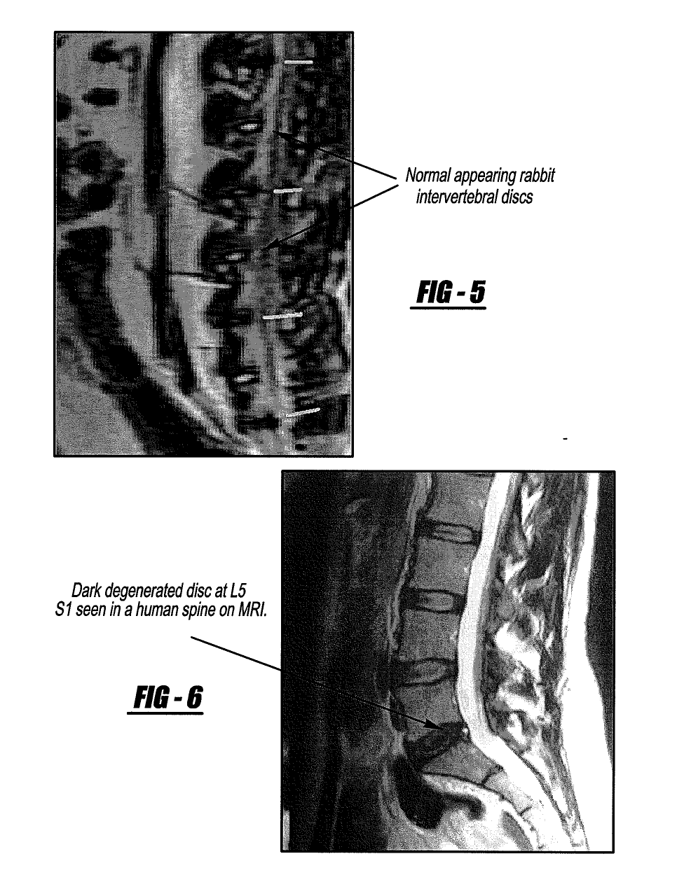

Once the disc 18 has been ruptured by the needle 12, and the degeneration process begins, its progress can be followed by magnetic resonant imaging (MRI) of the rabbit 10. FIG. 4 is a plan view of an MRI device 22 taking images of the rabbit 10 for this purpose. Other imaging device may also provide suitable images, such as image guidance technologies, computer tomography, tomograms, etc. FIG. 5 is an MRI of an animal rabbit model that has degenerated discs as a result of the process discussed above. FIG. 6 is an MRI of a human spine showing a degenerated disc at L₅S₁ in that it is similar to the degenerated disc in the rabbit model.

Once the disc degeneration model has been produced, then various experimentations and procedures can be used to treat the degenerated disc, which ultimately may be used on humans having degenerated discs. In one notochordal cells and chondrocytes that can produce cartilage. These cells can ultimately develop into a new disc. Additionally, various therapies can be implemented to treat the disc degeneration in the rabbit 10. For example, various drugs can be developed that can be experimentally used on the rabbit 10 to determine whether they have an effect on reducing the speed of the disc degeneration, reversing the disc degeneration, etc. Further, various therapies can be used to try to rehydrate the disc by injecting water holding drugs and other materials into the disc. Also, the rabbit disc degeneration model can be used for various other analyses and studies concerning disc degeneration including novel devices and instrumentation.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. A method for providing an InVivo model of disc degeneration, said method comprising:
providing a non-human animal; and
percutaneously inserting a needle into the animal so that the needle ruptures an annulus of a disc between vertebrae in the animal where the rupture induces disc degeneration.

2. The method according to claim 1 wherein providing a non-human animal includes providing a rabbit.

3. The method according to claim 1 wherein providing a non-human animal includes providing an animal selected from the group consisting of mice, rats, sheep and primates.

4. The method according to claim 1 wherein inserting a needle includes inserting a 16-gage needle into the animal.

5. The method according to claim 1 further comprising using an imaging device for visualizing the position of the needle relative to the disc of the animal so that the needle is properly inserted.

6. The method according to claim 5 wherein using an imaging device includes images using an imaging device selected from the group consisting of image guidance devices, x-ray imaging devices, computer tomography, tomograms and magnetic resonant imaging devices.

7. The method according to claim 5 wherein using an imaging device includes using a fluoroscopic x-ray imaging device.

8. The method according to claim 1 further comprising performing imaging of the animal after the needle has been inserted to visualize disc degeneration in the animal.

9. The method according to claim 8 wherein performing imaging includes performing magnetic resonant imaging.

10. The method according to claim 1 further comprising injecting stem cells into the ruptured disc that has been degenerated to regenerate the disc.

11. A method for providing an InVivo model of disc degeneration, said method comprising:
providing a rabbit;
percutaneously inserting a needle into the rabbit so that the needle ruptures an annulus of a disc between vertebrae in the rabbit where the rupture induces disc degeneration; and
performing imaging of the rabbit after the needle has been inserted to visualize the disc degeneration in the rabbit.

12. The method according to claim 11 further comprising using an imaging device for visualizing the position of the needle relative to the disc of the rabbit so that the needle is properly inserted.

13. The method according to claim 11 wherein performing imaging includes performing magnetic resonant imaging.

14. The method according to claim 11 further comprising injecting stem cells into the ruptured disc that has been degenerated to regenerate the disc.

15. A method for providing an InVivo model of disc degeneration, said method comprising:
providing a non-human animal;
intentionally damaging a disc between vertebrae in the animal so as to induce disc degeneration; and
injecting stem cells into the damaged disc to regenerate the disc.

16. The method according to claim 15 wherein intentionally damaging a disc includes inserting a needle into the disc.

17. The method according to claim 15 wherein providing a non-human animal includes providing a non-human animal selected from the group consisting of rabbits, mice, rats, sheep and primates.

18. The method according to claim 15 further comprising using an imaging device for visualizing the position of the needle relative to the disc of the animal so that the needle is properly inserted.

19. The method according to claim 15 further comprising performing imaging of the animal to visualize the degeneration and regeneration of the disc.

20. The method according to claim 19 wherein performing imaging includes performing magnetic resonant imaging.
